(19) United States
(12) Patent Application Publication
(10) Pub. No.: US 2021/0375411 A1
(43) Pub. Date: Dec. 2, 2021

(54) DIGITAL ADVANCE HEALTHCARE DIRECTIVE MANAGEMENT

(71) Applicant: Nneka Obiajulu Sederstrom, Prior Lake, MN (US)

(72) Inventor: Nneka Obiajulu Sederstrom, Prior Lake, MN (US)

(21) Appl. No.: 17/326,999

(22) Filed: May 21, 2021

Related U.S. Application Data
(60) Provisional application No. 63/029,969, filed on May 26, 2020.

(51) Int. Cl. G16H 10/60; G16H 10/20

(52) U.S. Cl. CPC G16H 10/60; G16H 10/20

(57) ABSTRACT

The present disclosure is directed to systems and methods for managing digital advance healthcare directives. In one aspect, a healthcare directive server is disclosed. The healthcare directive server communicates to a user computing device instructions and access information for a dynamic healthcare directive questionnaire. The healthcare directive server receives responses to the prompts from the user computing device, and records the responses in the healthcare directive data store, where the prompts are based on a patient account associated with the user computing device, and are accessed from the dynamic healthcare directive questionnaire.

### INTRODUCTION

Advance healthcare directives are legal documents describing a patient's wishes for their healthcare treatment if the patient is not able to make or express their wishes. The document could also be referred to as a living will, personal directive, advance directive, medical directive, or advance decision. Many advance healthcare directives also include information about a proxy that is selected by the patient to help make decisions for their healthcare. Some modern advance healthcare directives are digital and accessible online. Typical advance healthcare directives include a series of basic questions regarding healthcare decisions to be answered by the patient.

### SUMMARY

In general terms, the present disclosure is directed to systems and methods for managing digital advance healthcare directives. Various aspects are described in this disclosure, which include, but are not limited to, the following aspects.

In one aspect a healthcare directive server is disclosed. The directive server includes a dynamic healthcare directive questionnaire, a healthcare directive data store, at least one memory device; and at least one processing device, the at least one memory device comprising instructions. The instructions when executed by the at least one processing device, causes the processing device to communicate to a user computing device directive instructions and access information for the dynamic healthcare directive questionnaire. Where the directive instructions cause the user computing device to display prompts. The instructions further cause the processing device to receive responses to the prompts from the user computing device and record the responses in the healthcare directive data store. Where the prompts are based on a patient account associated with the user computing device, and are accessed from the dynamic healthcare directive questionnaire.

In another aspect a method for managing a dynamic health care directive is disclosed. The method comprising communicating to a user computing device instructions and access information for a dynamic healthcare directive questionnaire. Where the instructions cause the user computing device to display prompts. The method further comprising receiving responses to the prompts from the user computing device and recording the responses in a healthcare directive data store. Wherein the prompts are based on a patient account associated with the user computing device, and are accessed from the dynamic healthcare directive questionnaire.

In a further aspect a patient computing device is disclosed. The patient computing device comprising at least one memory device; and at least one processing device, the at least one memory device comprising instructions. The instructions when executed by the at least one processing device, causes the processing device to communicate to a healthcare directive server to provide a patient account ID and receive a dynamic healthcare directive questionnaire, and display prompts from the dynamic healthcare directive questionnaire. Where the prompts are based on the patient account ID. The instructions further cause the processing device to receive responses to the prompts and send the responses to the healthcare directive server.

### DESCRIPTION OF THE DRAWINGS

The following drawing figures, which form a part of this application, are illustrative of described technology and are not meant to limit the scope of the disclosure in any manner.

FIG. 1 shows a schematic diagram of an example healthcare environment in which the principles of the present invention may be employed.

FIG. 2 shows a schematic diagram of the healthcare directive server of FIG. 1

FIG. 3 is a flow chart of an example method for managing digital advance healthcare directives.

FIG. 4 shows example physical components of an example computing device usable in the healthcare environment of FIG. 1.

### DETAILED DESCRIPTION

Various embodiments of the present disclosure will be described in detail with reference to the drawings, wherein like reference numerals represent like parts and assemblies throughout the several views. Additionally, any examples set forth in this specification are not intended to be limiting and merely set forth some of many possible embodiments.

Broadly, the present disclosure is directed to systems and methods for managing digital advance healthcare directives. Patients can complete advance directives online using a dynamic questionnaire that provides prompts tailored to the patient. The patient can consult with healthcare professionals to discuss various aspects of their advance directive. The completed digital advance directive is easily accessed by the patient, medical professionals, and other interested parties.

The systems and methods disclosed for managing a digital advance healthcare directive provide several advantages. First, the digital advance healthcare directive is integrated to interface with a plurality of vendors (with different software platforms). This ensures that an up-to-date directive is provided in emergency situations. For example, in some emergency situations the patient may not be in a state to provide their wishes to a healthcare provider. The system and methods disclosed allow the provider to access an accurate directive using one or more vendors depending on what systems are available at the time of an emergency.

Additionally, life events (e.g. marriage, birth of a child, or change in medical circumstances) may change the patient's wishes for their directive. The systems and methods disclosed provide different ways automatically notify a patient to review their directive. In some examples this notification is done automatically at periodic times to ensure the directive is up-to-date. In other examples, this notification is sent to a patient automatically based on a scheduled procedure. The systems and methods manage a patient directive to ensure that the various vendors (using different technology platforms) are also updated to include the current directive for a patient.

The methods and systems disclosed further include integrations with one or more electronic medical record (EMR) systems to automate the input of standard patient information so that a patient does not need to enter the entirety of their information when completing their directive.

Next, the methods and systems disclosed herein provide a dynamic directive based on the patient. The dynamic directive improves the accuracy of the directive by selecting prompts based on the patient information. The methods and systems disclosed also allow a patient to communicate with an expert in real time while completing the directive to improve the patients understanding and comfort with the directive.

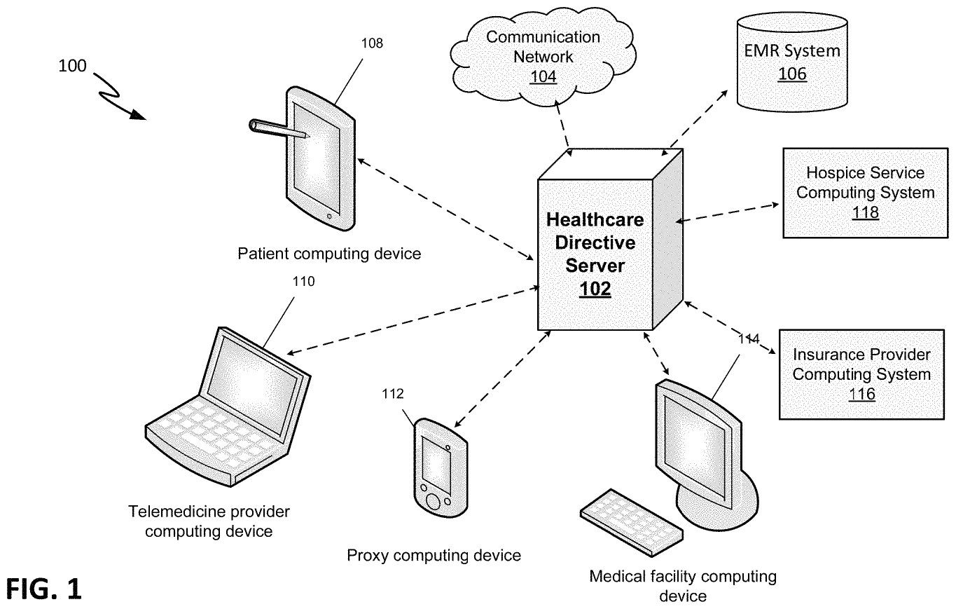

FIG. 1 is a schematic diagram of an example healthcare environment 100. The healthcare environment 100 operates to care for patients, including guiding the patients through preparing an advance healthcare directive. The healthcare environment 100 includes a healthcare directive server 102, a communication network 104, an electronic medical record system 106, a patient computing device 108, a telemedicine provider computing device 110, a proxy computing device 112, a medical facility computing device 114, an insurance provider computing system 116, and a hospice service computing system 118. Each component of this healthcare environment 100 communicates with each other via wired or wireless connections. In some embodiments, additional components can communicate with the healthcare environment 100 through the communication network 104. In some embodiments, some of the components shown in FIG. 1 are optional.

The healthcare directive server 102 operates to manage digital advance healthcare directives for a plurality of patients. Questionnaires are presented to the patients on the patient computing device 108. It will be appreciated that while a single patient computing device 108 is illustrated in FIG. 1, there are actually a plurality of devices 108 which may be used. More specifically, each patient reviewing and/or filling out the questionnaires may use one or more of their own devices 108 or may use a shared device 108.

Similarly, some embodiments include multiple healthcare directive servers 102. In these embodiments each of the multiple healthcare directive servers may be similar and provide similar functionality. Alternatively, in these embodiments, the multiple healthcare directive servers may perform specialized functions or specialized services. In some examples, the multiple healthcare directive servers may use a cloud computing technology to provide greater capacity, redundancy, or provide services from multiple geographic locations.

Additionally, a person of ordinary skill in the art will recognize that privacy and security is critical to the healthcare directive server 102. Accordingly, privacy and security may preclude certain server architecture solutions or may require a specific implementation. Additional services may also be required to ensure the privacy and security of the healthcare directive server 102.

Responses to the questionnaires are recorded in the electronic medical record (EMR) system 106. Patients can consult with medical professionals regarding questions about the healthcare directive. If the medical professionals are communicating with the patients remotely, they may operate a telemedicine provider computing device 110. After the digital advance healthcare directive is completed for a patient, a proxy can be notified on the proxy computing device 112. Medical professionals can access the patient's healthcare directive using the medical facility computing device 114 to inform decision-making regarding the patient's medical care. The patient's insurance provider can be notified that the patient has completed a healthcare directive via the insurance provider computing system 116. Additionally, the healthcare directive server 102 can communicate with a hospice service computing system 118 to provide patients with access to hospice resources in conjunction with completing digital advance directives.

One or more components of the healthcare environment 100 are in communication with each other via a communication network 104. The communication network 104 may include any type of wireless network, a wired network, or any communication network known in the art. For example, wireless connections can include cellular network connections and connections made using protocols such as 802.11a, 802.11g, 802.11n, and/or 802.11ac.

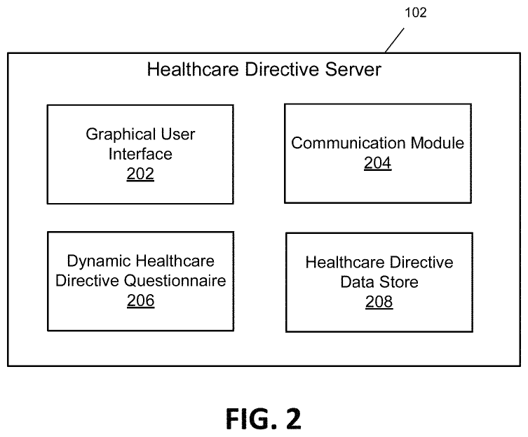

FIG. 2 is a more detailed schematic diagram of the healthcare directive server 102 of FIG. 1. The healthcare directive server 102 manages healthcare directive questionnaires, patient's completed directives, and communication of the directives to the relevant parties. The healthcare directive server 102 includes at least one processing device and at least one memory device (not shown). The memory device includes instructions to operate a graphical user interface 202, a communication module 204, and a dynamic healthcare directive questionnaire 206 when executed by the processing device. The memory device also includes a healthcare directive data store 208.

The graphical user interface (GUI) 202 operates to present a visual display for computing device users to access, enter, edit, and delete healthcare directive information. In some embodiments, the GUI 202 presents a dynamic healthcare directive questionnaire to a patient or to an individual assisting a patient in preparing a healthcare directive. In some embodiments, the individual is a medical professional such as a physician, nurse, ethicist, or designee. In some embodiments, the individual is a proxy or a loved one. Responses to the questionnaire are received at the GUI 202 through inputs provided at a computing device.

In some embodiments, the GUI 202 receives input from a computing device requesting to view a completed healthcare directive. In some embodiments, the computing device is a telemedicine provider computing device 110, a proxy computing device 112, or a medical facility computing device 114 accessing a patient's healthcare directive in order to counsel the patient or to aid in making decisions for the patient's healthcare. The patient's digital healthcare directive is then displayed on the GUI 202.

In some embodiments, a patient may utilize the GUI 202 to edit or delete their digital healthcare directive. For example, the GUI 202 receives input from a patient computing device 108 of the patient editing, or deleting their digital healthcare directive. In other examples, the inputs are received by a proxy computing device 112 where the inputs are submitted by a proxy for the patient.

The communication module 204 operates to manage communication of healthcare directive information to and from the healthcare directive server 102. In some embodiments, the communication module 204 communicates with patient computing devices 108, telemedicine provider computing devices 110, proxy computing devices 112, and medical facility computing devices 114. In some embodiments, dynamic healthcare directive questionnaires 206 are communicated to patient computing devices 108 and telemedicine provider computing devices 110 and completed healthcare directives are received in return. In some embodiments, the communication module 204 manages requests for information received from EMR systems 106, insurance provider computing systems 116, and medical facility computing devices 114. In some embodiments, the communication module 204 directs communication of patient healthcare directives to the requesting computing system for display on a screen of a computing device. In some embodiments, the communication module 204 coordinates communication between patients and medical professionals regarding appointments, questions about healthcare directives, information about a patient's health, and information in a patient's healthcare directive.

The dynamic healthcare directive questionnaire 206 operates to present to a patient (or individual aiding a patient) a series of prompts or questions regarding various aspects of the patient's healthcare wishes. Further details regarding an example dynamic questionnaire are provided below.

The healthcare directive data store 208 operates to store information regarding patients' healthcare directives. In some embodiments, a record of the patients that have completed a digital healthcare directive is kept. In some embodiments, partially completed digital healthcare directives are stored and a patient can later access their digital healthcare directive to complete it. In some embodiments, completed digital healthcare directives are stored for later access by medical facilities, proxies, etc.

Preferably the stored healthcare directives are securely stored in data store 208 and may be accessed in whole or in part only by authorized personnel (described in more detail below). Accordingly, devices and systems which secure the data in the data store 208 are preferably provided. For example, password protection, multi-factor authentication, and block chain type systems may be used among others.

Although only one healthcare directive data store 208 is illustrated in FIG. 2, some examples include multiple healthcare directive data stores 208. In these examples, the multiple healthcare directive data stores 208 can be configured to provide a modular, scalable, and redundant storage. In some examples, a cloud computing technology is used.

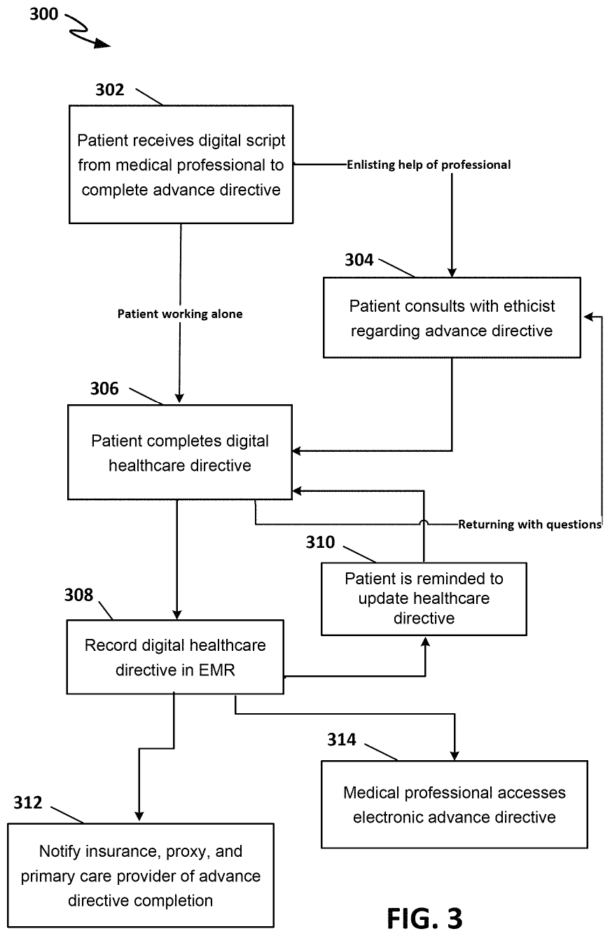

FIG. 3 is an example flowchart of a method 300 for managing digital advance healthcare directives. In some embodiments, this method 300 is performed by one or more components in the healthcare environment 100 of FIG. 1. In particular, the healthcare directive server 102 performs many aspects of the method 300.

At operation 302, the patient receives a digital script from a medical professional to complete an advance directive. In some embodiments, a patient's need for an advance directive is discussed with a medical professional during an appointment. The appointment could be in-person or online (telemedicine). In some embodiments, the medical professional can add a note to a patient's EMR indicating that the patient needs to complete an advance directive and include a link to an online digital healthcare directive. In some embodiments, a patient that does not already have an advance directive recorded in their EMR may be asked when they check-in for an appointment or make an appointment if they would like to speak to a medical professional about completing a healthcare directive.

In some embodiments, the medical professional could write a digital script for the patient to visit an ethicist or designee to discuss their advance directive. The patient could then be assisted in making an appointment with the ethicist or designee through online scheduling or the aid of an administrative professional at the healthcare facility the patient is visiting. If the patient enlists the help of a professional to aid in completing the advance directive, the method proceeds to operation 304.

In some embodiments, a patient accesses a digital advance directive questionnaire without being provided with a script or prompt from a healthcare professional. In some embodiments, the patient can download a software application or access a web service to complete a directive.

At operation 304, the patient consults with an ethicist or designee (or other medical professional) regarding their advance directive. The consultation could be done in person or online (telemedicine). In some embodiments, the consultation is performed through a digital communication tool allowing for face-to-face conversation between the ethicist or designee and the patient (or patient's proxy). In some embodiments, the patient could simply have a phone conversation with an ethicist or designee regarding questions that the patient has regarding their health directive. In some embodiments, a patient's physician can consult directly with the ethicist or designee to answer a patient's questions or to guide a patient in completing a directive.

After this consultation, the method proceeds to operation 306. However, in some embodiments, operation 306 occurs at the same time as operation 304. In some embodiments, the ethicist or designee assists in the completion of the healthcare directive based on discussions with the patient about their healthcare wishes.

If the patient is working alone on their advance directive, the method proceeds to operation 306. Note that the advance directive could be completed by the patient (if the patient is an adult), a parent or guardian of the patient (if the patient is under 18 years of age), or a proxy individual appointed by the patient or designated legal guardian to assist with completing the questionnaire. At operation 306, the patient completes a digital healthcare directive. In some embodiments, the patient utilizes a patient computing device such as a smartphone, tablet, laptop, or desktop computer to complete a dynamic questionnaire. Details of the questionnaire are provided below.

In some embodiments, if the patient has questions while completing their directive, they can make an appointment to consult with an ethicist or designee or other professional and the method returns to operation 304.

At operation 308, the patient's digital healthcare directive is recorded in the patient's electronic medical record (EMR). In some embodiments, the patient uploads a completed digital healthcare directive to their EMR through a patient portal online. In some embodiments, if the patient has logged into a patient portal linked to their EMR, the digital healthcare directive will be automatically saved with the patient's EMR.

In some examples, the data may be anonymized and collected to train a model. In these examples, before collecting and anonymizing the data the patient is asked whether they consent to having their anonymized data used to train a model. The model is trained to analyze and make insights on the dynamic directive. In these examples, the model is used to further enhance the dynamic directive. For example, the model may select certain prompts based a patient profile or specific follow up prompts. A model may also be trained to recommend the patient receive a second opinion based on the received responses to directive questions. In some examples, the model is trained using machine-learning, artificial intelligence, or business intelligence.

Periodically, the method proceeds to operation 310 and the patient is reminded to update their healthcare directive. In some embodiments, reminders are communicated to the patient electronically. For example, the patient could receive emails triggered by their EMR indicating that the patient should review their healthcare directive and potentially update the directive. In some embodiments, the patient's EMR could be flagged to indicate to a medical professional to discuss updating the patient's healthcare directive at the patient's next appointment. In some embodiments, the frequency of the reminders is determined based on the patient's age.

In some embodiments, after the patient's healthcare directive is completed, one or more parties can be notified electronically. At operation 312, one or more of an insurance, proxy, and/or primary care provider are notified of advance directive completion.

In some embodiments, after a proxy is notified that they have been selected by a patient, the proxy is presented with multiple options. One option presents the proxy with information to contact the patient's ethicist, designee and/or medical professional to address any questions or concerns that the proxy has. Another option allows the proxy to decline their appointment as the patient's proxy. This could be because the person named to be the patient's proxy does not feel that they can be a good decision maker or because they do not want the responsibility. The option could present an “opt out” button for the proxy to decline being the named decision maker, which triggers a notification to a patient to identify an alternative. In some embodiments, the patient has appointed a primary proxy decision maker and a back-up proxy decision maker. In such embodiments, if the primary proxy opts out, the back-up proxy is automatically notified. The back-up proxy can also be presented with an option to opt-out.

At operation 314, one or more medical professionals can access the patient's electronic advance directive. In some embodiments, it is accessed from the patient's EMR. In some embodiments, the directive could be stored on a personal computing device of the patient such as a smartphone. In some embodiments, the directive is stored in a digital database of directives managed by an organization such as a state-sponsored organization.

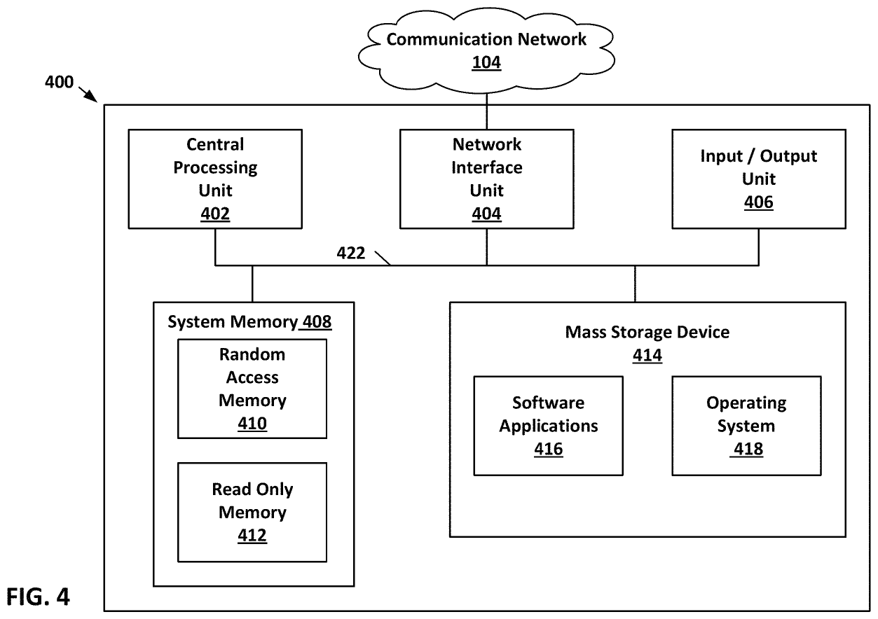

FIG. 4 is a block diagram illustrating an example of the physical components of a computing device 400. The computing device 400 could be implemented in various aspects of the healthcare environment 100, such as the patient computing device 108, telemedicine provider computing device 110, and proxy computing device 112. Components of the computing device 400 can also be incorporated into other devices and systems described herein, such as the insurance provider computing system 116 and EMR system 106.

In the example shown in FIG. 4, the computing device 400 includes at least one central processing unit (“CPU”) 402, a system memory 408, and a system bus 422 that couples the system memory 408 to the CPU 402. The system memory 408 includes a random access memory (“RAM”) 410 and a read-only memory (“ROM”) 412. A basic input/output system that contains the basic routines that help to transfer information between elements within the computing device 400, such as during startup, is stored in the ROM 412. The computing system 400 further includes a mass storage device 414. The mass storage device 414 is able to store software instructions and data.

The mass storage device 414 is connected to the CPU 402 through a mass storage controller (not shown) connected to the system bus 422. The mass storage device 414 and its associated computer-readable storage media provide non-volatile, non-transitory data storage for the computing device 400. Although the description of computer-readable storage media contained herein refers to a mass storage device, such as a hard disk or solid state disk, it should be appreciated by those skilled in the art that computer-readable data storage media can include any available tangible, physical device or article of manufacture from which the CPU 402 can read data and/or instructions. In certain embodiments, the computer-readable storage media comprises entirely non-transitory media.

Computer-readable storage media includes volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the computing device 400.

According to various embodiments, the computing device 400 can operate in a networked environment using logical connections to remote network devices through a communication network 104, such as a wireless network, the Internet, or another type of network. The computing device 400 may connect to the communication network 104 through a network interface unit 404 connected to the system bus 422. It should be appreciated that the network interface unit 404 may also be utilized to connect to other types of networks and remote computing systems. The computing device 400 also includes an input/output controller 406 for receiving and processing input from a number of other devices, including a touch user interface display screen, or another type of input device. Similarly, the input/output controller 406 may provide output to a touch user interface display screen or other type of output device.

As mentioned briefly above, the mass storage device 414 and the RAM 410 of the computing device 400 can store software instructions and data. The software instructions include an operating system 418 suitable for controlling the operation of the computing device 400. The mass storage device 414 and/or the RAM 410 also store software instructions, that when executed by the CPU 402, cause the computing device 400 to provide the functionality discussed in this document.

### Example System Setup

The following provides information for one possible setup of a digital directive system. Other embodiments and implementations are possible.

Digital Directive Setup

As discussed above, in many embodiments the dynamic directive provides seamless communication between patient, proxy, provider, payer, ethicist or designee. Additionally, the dynamic directive is often configured to interface with an EMR and/or a hospice connection. Each of the various users and/or systems must maintain certain data protection requirements. In some examples, the users must agree to upholding confidentiality before using the digital directive. Additionally, the users can be required to agree to upholding confidentiality rules before entering the app or setting up an account.

Different users may have different levels of access. For example, a patient and a provider may have complete access to the dynamic directive. In contrast a proxy does not have access to edit or change the dynamic directive. For example, the proxy may only have access to provide the directive to a healthcare provider. In some examples, the proxy is prompted to agree to serve as a decision maker for a patient. If the primary proxy declines to serve as a decision maker, then a secondary proxy is notified. In some examples, a payer only has access related to whether the patient has completed the dynamic directive or if the patient makes any revisions or updates to the dynamic directive. In some examples, an ethicist or designee has access to the dynamic directive in a read only format. Accordingly, the ethicist or designee only has access necessary to provide support and clarification for a patient and/or a proxy.

System Integration Needs

In some embodiments dynamic directive is integrated with systems from multiple vendors. Accordingly, the dynamic directive may include an application programming interface (API) layer to facilitate these integrations. For example, the application may have an API layer which allows the various venders to request different features which the application may provide a response to properly authenticated parties. Similarly, the application may interface with other vendors API's. For example, the application may interface with an EMR system to store the completed directive.

In some embodiments the dynamic directive is integrated with one or more electronic medical record (EMR) systems. For example, the dynamic directive is uploaded to an EMR system with a timestamp. Additionally, the dynamic directive is integrated with a patient portal. The patient portal may have a folder which gives the patient easy access to the dynamic directive. The patient portal may also send updates to the patient. For example, the patient portal may notify the patient to discuss the dynamic directive with the patient's primary care physician at a clinical visit.

In some examples, the patient portal may prompt a patient to questions related to sharing or discussing the dynamic directive with a new or existing specialist. For example, the patient may be asked to share the dynamic directive through the patient portal with other clinicians. In some examples a pop-up notice is displayed for the patient for when a new appointment is made with a new clinician to see if the patient is interested in sharing the dynamic directive with the new clinician.

In some examples, the EMR system for the primary care physician may be different from the EMR system used by a specialist. The dynamic directive may be configured to interface with a plurality of different EMR systems. In some examples, some sections of the dynamic directive are automatically populated with information saved in the EMR system. For example, the EMR system can populate the personal data section for a patient.

In some embodiments the primary physician clinic sends reminders through the EMR system and/or the patient portal to remind the patient to review the dynamic directive at or before a clinic visit. These reminders may be automated to be sent out at a predetermined time. For example, the reminders to review the dynamic directive may be sent every 6 months or every year. In some examples, a record in the EMR system is updated when the patient updates the dynamic directive and the primary physician clinic is notified about the update.

The system is also be configured to send additional notifications to specific users. These notifications, in some examples, are sent over email or text message. Other secure messaging applications can also be used. In one example the system may send a secure email notification to a proxy based on different events. For example, the message is sent to the proxy to prompt the proxy to save the dynamic directive on the proxy's personal account under the patient's name. In some examples, the proxy has a professional account and a personal account and may receive notifications from both accounts. In another example the proxy receives notifications of any updates or changes to the patient's dynamic directive.

Similarly, the ethicist or designee may receive notifications from the system. For example, the ethicist or designee may receive a message with the patient's dynamic directive before meeting with the patient. In some examples, this message is removed after the ethicist or designee meets with the patient. The ethicist or designee may also receive notifications when the patient makes updates or changes to their dynamic directive.

The ethicist (or designee) and proxy may have separate accounts with their own dynamic directives. This allows these users to keep their personal and professional accounts separate. In some examples, the professional and personal accounts are accessed from the same part of the application. For example, a user-interface may include one selection for “Digital Directives” which presents a display with a “My Directive” selection linking to a personal directive and a “Proxy” selection linking to a list of directives the individual is proxy to. Each selection may include selections for accessing related personal or proxy services. Alternatively, professionals can utilize a separate software application. For example, a professional application and a personal application.

In some examples, the application prompts the patient or proxy to get a second opinion after meeting with an ethicist or designee. The application may select a physician or other expert for the second opinion based on the patient's demographic information. A patient may wish to get a second opinion from a physician with a similar demographic background. For example, a patient may receive a request asking if the patient wishes to speak to a physician of the same race to discuss the dynamic directive. Providing a second opinion in this manner may improve health equity by enhancing the accuracy of the dynamic directive and improving clinical outcomes for the patient.

In some examples, the application may present scenarios to the user which allows a user to understand how the dynamic directive would be used in different scenarios. The patient may then update the dynamic directive based on the second opinion or on the presented scenarios.

In some examples, the application may generate a machine-readable code which when scanned by a device transfers the patient ID associated with the dynamic directive to an approved user. In some examples the machine-readable code is a QR code. In some examples, when the code is scanned by a properly authenticated device a message is displayed on the device informing the healthcare provider that the patient has a directive.

In some examples, the mobile computing device may detect that the patient is in a critical condition, using sensors on a phone or connected smart device like a smart watch. After detecting that the patient is in critical condition the phone may automatically display a patient ID number or a QR code associated with the patient ID. Similarly, the mobile application may automatically ping the EMR system to notify the healthcare provider that the patient has a dynamic directive.

In other examples, the application may interface with an emergency contact feature in a mobile computing device. For example, some mobile computing devices include a feature to contact an emergency contact without unlocking the phone. The digital directive application may interface which such a feature to allow a user to contact a directive contact (e.g. a proxy, or designee) without unlocking the phone when the device owner is experiencing a medical emergency.

In other examples, a care provider may have a QR code which facilitates the sign-up of a new patient to use the dynamic directive. In this manner a doctor may instruct the patient to complete the questionnaire after being directed to application.

### Questionnaire

The following outlines a series of prompts that are presented to a patient in a dynamic questionnaire for completing a dynamic advance directive. Note that the questionnaire could be completed by the patient (if the patient is an adult), a parent or guardian of the patient (if the patient is under 18 years of age), a medical professional caring for the patient, or a proxy individual appointed by the patient to assist with completing the questionnaire. This is just one example of the information that a patient might be requested to provide. Other embodiments and implementations are possible.

In some examples, the prompts provided to the patient are based on the patient ID or an account associated with the patient. Similarly, the prompts presented may be based on the patient's medical history or demographic information.

Introduction Statement

The Digital Directive is a chance for you to take control of your right to decide about your care based on your values. It is designed to help your family, friends, and providers know what is important to you when you are not able to speak for yourself. You have the option to revisit, revise, and cancel this directive at any time. Your voice is what matters.

Personal Data
Demographic data
   i. Name
   ii. Address
   iii. Married/Partner
   iv. Gender Identity
   v. Race/Ethnicity
   vi. Optional: Religion
   vii. DOB
   viii. Primary doc info
   ix. Subspecialist physician info
DPA/medical decision maker
   i. Name and contact info
   ii. Relationship to account owner
   iii. Are they aware of being the DPA
Values and quality of life
   i. List of values that you hold dear (click all)
      1. Engaging with family and/or friends
      2. Being active
      3. Independence
      4. Truthfulness
      5. Frank and Direct communication
      6. Ability to work
      7. Travel and leisure activities
      8. Engaging in pastimes and/or hobbies (Free text to list top 3)
   ii. Would you want your quality of life to be a factor in decision making and if so, what values do you hold as the highest
      1. Ability to communicate effectively
      2. Ability to walk or be mobile without assistants or with assistance
      3. Recognizing and engaging with family and/or friends in meaningful ways
      4. Ability to see, hear, touch and/or feel
      5. Ability to care for my personal body needs
      6. Be able to stay in my home for as long as I can
   iii. Things about me I want my caregivers to know:
      1. Free text about hobbies and other things they may want caregivers to know
      2. Opportunity to be specific on values and quality of life indicators
   iv. I want my doctors to only provide the medically appropriate options to achieve my values and goals to my DPA. (filling out directive is optional if this is checked)

My Advance Directive and Wishes
If I am in a permanently vegetative state (coma), these are my wishes (to include starting and/or stopping of interventions):
   i. Vent yes/no
   ii. Feeding tube yes/no
   iii. Dialysis y/n
   iv. ECMO/LVAD/adv heart devices yes/no
   v. Comfort care medication/pain meds yes/no
   vi. Autopsy yes/no
   vii. Accept time limited trial of up to 2 weeks on any of these features yes/no
   viii. Other specific requests
   ix. Hospice referral
If I have a terminal or end stage disease or condition defined as having less than 6 months of life left, these are my wishes (to include starting and/or stopping of interventions):
   i. Vent yes/no
   ii. Feeding tube yes/no
   iii. Dialysis y/n
   iv. ECMO/LVAD/adv heart devices yes/no
   v. Comfort care medication/pain meds yes/no
   vi. Autopsy yes/no
   vii. Accept time limited trial of up to 2 weeks on any of these features yes/no
   viii. Hospice referral
I am willing to engage in any aggressive interventions for a limited time trial. Continuation of these interventions must be based on clear medical evidence of my significant clinical improvement. If there are no evidence to justify continuing, I wish to have these interventions stopped. Yes/No
Other specific requests

Religious Values
Check boxes for religious group with free text option to describe wishes

Witnessed and signed through electronic means/e-sign/notification to DPA

I would like to speak with a health professional about my advance directive
In person or telemedicine visit with Primary care doc
Telemedicine visit with an Ethicist or designee

After completing the dynamic directive, an email can be sent to the patient's primary care provider, any relevant specialists, DPA, and the patient's EMR. Following completion, reminders are sent to the patient on a scheduled basis. For individuals under 65 years of age, yearly notifications are sent requesting for the patient to renew or reaffirm their directive. For individuals 65 and over, the notifications are sent every 6 months.

Although various embodiments are described herein, those of ordinary skill in the art will understand that many modifications may be made thereto within the scope of the present disclosure. Accordingly, it is not intended that the scope of the disclosure in any way be limited by the examples provided.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

1. A healthcare directive server comprising:
a dynamic healthcare directive questionnaire;
a healthcare directive data store;
at least one memory device; and
at least one processing device, the at least one memory device comprising instructions that, when executed by the at least one processing device, causes the processing device to:
communicate to a user computing device directive instructions and access information for the dynamic healthcare directive questionnaire, where the directive instructions cause the user computing device to display prompts;
receive responses to the prompts from the user computing device; and
record the responses in the healthcare directive data store;
wherein the prompts are based on a patient account associated with the user computing device, and are accessed from the dynamic healthcare directive questionnaire.

2. The healthcare directive server of claim 1, wherein the instructions when executed by the at least one processing device, further cause the processing device to:
communicate with an electronic medical records system to access at least one of patient medical information and demographic information.

3. The healthcare directive server of claim 2, wherein the prompts are further based on at least one of the medical information and the demographic information.

4. The healthcare directive server of claim 1, wherein the instructions when executed by the at least one processing device, further cause the processing device to:
communicate follow up prompts to the user computing device based on the responses.

5. The healthcare directive server of claim 1, wherein the instructions when executed by the at least one processing device, further cause the processing device to:
provide electronic access to at least one of an ethicist, designee, and a physician for questions through one of telemedicine, messaging, or online appointment scheduling.

6. The healthcare directive server of claim 1, wherein at least one of the prompts requests at least one of medical information and demographic information.

7. The healthcare directive server of claim 1, wherein the responses are provided to one or more electronic medical record systems.

8. The healthcare directive server of claim 7, wherein the one or more electronic medical record systems automatically provides the responses to a medical provider computing device when the electronic medical record system receives an emergency message associated with the patient account.

9. The healthcare directive server of claim 1, wherein the patient account is associated with a proxy account, wherein, a device associated with the proxy account receives notifications when the responses are updated.

10. The healthcare directive server of claim 1, wherein the instructions when executed by the at least one processing device, further cause the processing device to:
send a message periodically to the user computing device to generate a notification requesting an update to the digital advance healthcare directive questionnaire.

11. A method for managing a dynamic health care directive, the method comprising:
communicating to a user computing device instructions and access information for a dynamic healthcare directive questionnaire, where the instructions cause the user computing device to display prompts;
receiving responses to the prompts from the user computing device; and
recording the responses in a healthcare directive data store;
wherein the prompts are based on a patient account associated with the user computing device, and are accessed from the dynamic healthcare directive questionnaire.

12. The method of claim 11, the method further comprising:
communicating with an electronic medical records system to access at least one of patient medical information and demographic information.

13. The method of claim 12, wherein the prompts are further based on at least one of the medical information and the demographic information.

14. The method of claim 11, the method further comprising:
communicating follow up prompts to the user computing device based on the responses.

15. The method of claim 11, the method further comprising:
providing electronic access to at least one of an ethicist, designee and a physician for questions through one of telemedicine, messaging, or online appointment scheduling.

16. A patient computing device comprising:
at least one memory device; and
at least one processing device, the at least one memory device comprising instructions that, when executed by the at least one processing device, causes the processing device to:
communicate to a healthcare directive server to provide a patient account ID and receive a dynamic healthcare directive questionnaire;
display prompts from the dynamic healthcare directive questionnaire, wherein the prompts are based on the patient account ID;
receive responses to the prompts; and
send the responses to the healthcare directive server.

17. The patient computing device of claim 16, wherein the instructions further cause the processing device to:
receive follow up prompts from the healthcare directive server based on the responses.

18. The patient computing device of claim 16, wherein the instructions further cause the processing device to:
establish a communication channel with at least one of an ethicist, designee, and a physician to discuss the dynamic healthcare directive questionnaire.

19. The patient computing device of claim 16, wherein the patient computing device scans a machine-readable code to initiate the communication with the healthcare directive server.

20. The patient computing device of claim 16, wherein the patient computing device generates a machine-readable code which when scanned allows a health care provider device to receive the patient account ID and the responses.
